(19) United States
(12) Patent Application Publication
(10) Pub. No.: US 2019/0060501 A1
(43) Pub. Date: Feb. 28, 2019

(54) DEODORIZING DEVICE

(71) Applicant: Corey Calvin Arnold, Spruce Grove (CA)

(72) Inventor: Corey Calvin Arnold, Spruce Grove (CA)

(21) Appl. No.: 15/684,880

(22) Filed: Aug. 23, 2017

(51) Int. Cl. A61L 9/012; A61L 9/013; A61L 9/014; A61L 2/24; A24F 13/06

(52) U.S. Cl. CPC A61L 9/012; A61L 9/013; A24F 13/06; A61L 2/24; A61L 9/014

(57) ABSTRACT

A deodorizing device having a cylindrical body having a first end and a second end, a mouthpiece, a first cartridge having a first end and a second end, a second cartridge, a first filter, a second filter, a plurality of seals, a screen. The first cartridge holds charcoal and the second cartridge holds an extract for masking and eliminating the scent of marijuana when smoking.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of smoking accessories and more specifically relates to a deodorizing smoking accessory, entitled ‘lowketoke’.

### 2. Description of Related Art

Smoking is a practice in which a substance is burned and the resulting smoke breathed in to be tasted and absorbed into the bloodstream. Most commonly the substance is the dried leaves of the tobacco plant which have been rolled into a small square of rice paper to create a small, round cylinder called a “cigarette”. Smoking is primarily practiced as a route of administration for recreational drug use because the combustion of the dried plant leaves vaporizes and delivers active substances into the lungs where they are rapidly absorbed into the bloodstream and reach bodily tissue. Smoking is one of the most common forms of recreational drug use. The smell of smoke is often undesirable and difficult to mask. A suitable solution is desired.

U.S. Pub. No. 2017/0042223 to Howard Park relates to a device and method for containing and filtering exhaled smoke. The described device and method for containing and filtering exhaled smoke includes a device and method for containing and filtering exhaled smoke. When a smoker exhales smoke into the mouthpiece, the smoke is initially stored in a bag or bellows. Pressure on the bag or bellows, provided by the material itself, gravity, or a spring, forces the smoke through a filter before being released into the surrounding air.

### BRIEF SUMMARY OF THE INVENTION

In view of the foregoing disadvantages inherent in the known smoking accessories art, the present disclosure provides a novel deodorizing device. The general purpose of the present disclosure, which will be described subsequently in greater detail, is to provide users with a deodorizer for marijuana smoke.

A deodorizing device is disclosed herein. The deodorizing device includes a cylindrical body having a first end and a second end, a mouthpiece, a first cartridge having a first end and a second end, a second cartridge, a first filter, a second filter, a plurality of seals, a screen. The mouthpiece is removably attached to the first end of the cylindrical body. The first cartridge holds charcoal and the second cartridge holds an extract. The extract may include an organic fruit extract for eliminating and deodorizing smoke. The deodorizing device is configured to deodorize an odor and eliminate the smell of medical marijuana while smoking using charcoal and extract scents.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a deodorizing device, constructed and operative according to the teachings of the present disclosure.

FIG. 1 is a perspective view of the deodorizing device during an ‘in-use’ condition, according to an embodiment of the disclosure.

FIG. 2 is a perspective view of the deodorizing device of FIG. 1, according to an embodiment of the present disclosure.

FIG. 3 is a perspective view of the deodorizing device of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a smoking accessory and more particularly to a deodorizing device as used to improve the deodorizing and odor removal of marijuana smoke.

Generally, the deodorizing device is a deodorizer for marijuana users that want to cover or mask the smell of the smoke. The device includes scents and interchangeable parts with a fan for better air flow and caps to keep the deodorizer fresh. A user blows through the device and it gets rid of the smoke and smell leaving a scent of multiple fruity or spicy options.

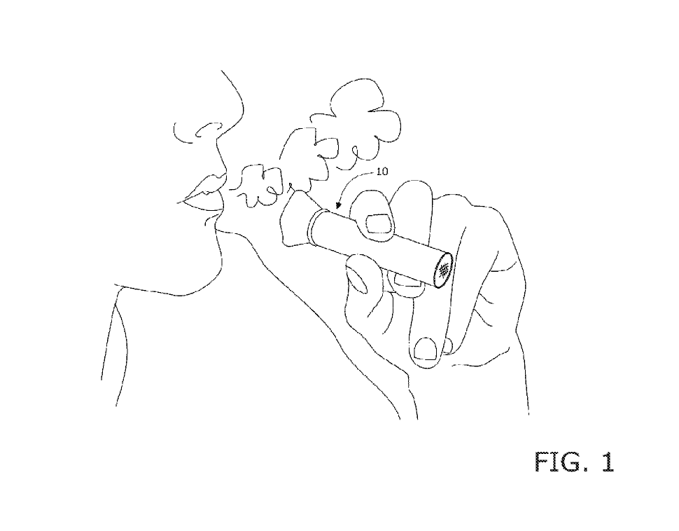

Referring now more specifically to the drawings by numerals of reference, there is shown in FIGS. 1-3, various views of a deodorizing device 10. FIG. 1 shows a deodorizing device 10 during an ‘in-use’ condition, according to an embodiment of the present disclosure. As illustrated, the deodorizing device 10 may include a cylindrical body 12 having a first end 14 and a second end 16, a mouthpiece 18, a first cartridge 20 having a first end 22 and a second end 24, a second cartridge 25, a first filter 30, a second filter 32, a plurality of seals, and a screen 34.

The mouthpiece 18 is removably attached to the first end 22 of the cylindrical body 12. The first cartridge 20 may be housed in the cylindrical body 12. The second cartridge 25 includes a first end 26 and a second end 28; the second cartridge 25 being housed in the cylindrical body 12 and removably coupled to the first cartridge 20. The first filter 30 is housed in the cylindrical body 12 and able to be removably attached to the first end 22 of the first cartridge 22. The second filter 32 is housed in the cylindrical body 12 and being removably attached to the second end of the first cartridge 24. The plurality of seals are housed in the cylindrical body 12. The screen 34 may be removably coupled to the second end of the cylindrical body 12. The deodorizing device 10 is configured to deodorize an odor and effectively eliminate the smell of marijuana while smoking.

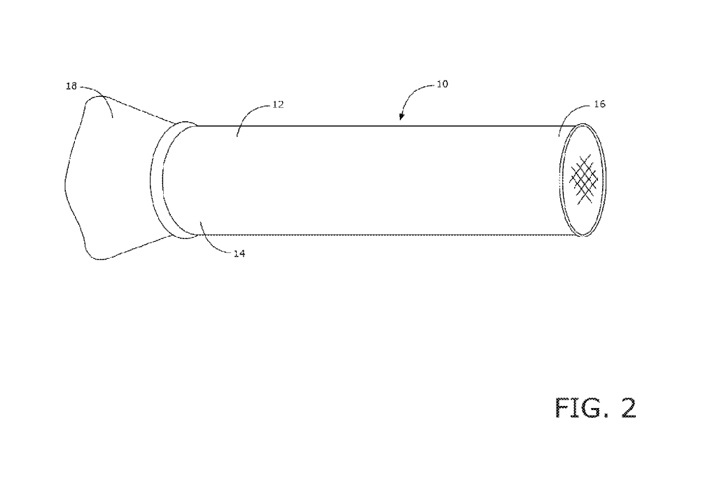

FIG. 2 shows a perspective view of the deodorizing device 10 of FIG. 1, according to an embodiment of the present disclosure. As above, the deodorizing device 10 may include means for masking the smell of medical marijuana while smoking. The deodorizing device 10 may comprise the cylindrical body 12, the mouthpiece 18, the first cartridge 20, the second cartridge 25, the first filter 30, the second filter 32, the plurality of seals, and the screen 34. The first cartridge 20 holds charcoal and the second cartridge 25 holds an extract. The extract may include an organic fruit extract. In a preferred embodiment, the charcoal is activated charcoal. The activated charcoal is in powder-form. The second cartridge 25 is removably coupled to the first cartridge 20 by a first threading means 36 or other suitable fastening means.

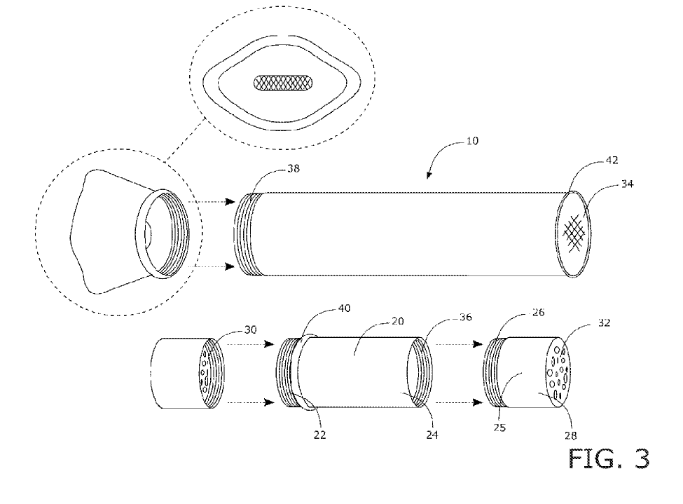

FIG. 3 shows a perspective view of the deodorizing device 10 of FIG. 1, according to an embodiment of the present disclosure. As above, the deodorizing device 10 may include the cylindrical body 12, the mouthpiece 18, the first cartridge 20, the second cartridge 25, the first filter 30, the second filter 32, the plurality of seals, and the screen 34. The first cartridge 20 and the second cartridge 25 are replaceable. The first filter 30 and the second filter 32 are made of foam. The mouthpiece 18 is removably attached to the first end 14 of the cylindrical body 12 by a second threading means 38. The mouthpiece 18 may conform to the shape of lips.

The plurality of seals includes a first seal 40 and a second seal 42. The first seal 40 is removably attached to the first end of the first cartridge 22. The second seal 42 is removably attached to the screen 34. The plurality of seals may be made of rubber. The plurality of seals are O-ring seals or other suitable sealing means.

The deodorizing device 10 is configured to deodorize an odor and eliminate (and/or effectively mask or minimize or change) the smell of medical marijuana while smoking. Certain embodiments may include a fan. Other embodiments may not include a fan.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A deodorizing device, said deodorizing device comprising:
a cylindrical body having a first end and a second end;
a mouthpiece, said mouthpiece is removably attached to said first end of said cylindrical body;
a first cartridge having a first end and a second end, said first cartridge being housed in said cylindrical body;
a second cartridge, said second cartridge having a first end and a second end, said second cartridge being housed in said cylindrical body and removably coupled to said first cartridge;
a first filter, said first filter being housed in said cylindrical body and being removably attached to said first end of said first cartridge;
a second filter, said second filter being housed in said cylindrical body and being removably attached to said second end of said first cartridge;
a plurality of seals said plurality of seals being housed in said cylindrical body;
a screen, said screen being removably coupled to said second end of said cylindrical body; and
wherein said deodorizing device is configured to deodorize an odor.

2. The deodorizing device of claim 1, wherein said first cartridge holds charcoal.

3. The deodorizing device of claim 1, wherein said second cartridge holds an extract.

4. The deodorizing device of claim 3, wherein said extract is an organic fruit extract.

5. The deodorizing device of claim 2, wherein said charcoal is activated charcoal.

6. The deodorizing device of claim 5, wherein said activated charcoal is in powder-form.

7. The deodorizing device of claim 1, wherein said second cartridge is removably coupled to said first cartridge by a first threading means.

8. The deodorizing device of claim 1, wherein said first and said second cartridges are replaceable.

9. The deodorizing device of claim 1, wherein said first and said second filters are made of foam.

10. The deodorizing device of claim 1, wherein said mouthpiece is removably attached to said first end of said cylindrical body by a second threading means.

11. The deodorizing device of claim 1, wherein said plurality of seals includes a first seal and a second seal.

12. The deodorizing device of claim 11, wherein said first seal is removably attached to said first end of said first cartridge.

13. The deodorizing device of claim 11, wherein said second seal is removably attached to said screen.

14. The deodorizing device of claim 1, wherein said plurality of seals are made of rubber.

15. The deodorizing device of claim 1, wherein said plurality of seals are O-ring seals.

16. The deodorizing device of claim 1, wherein said odor includes medical marijuana.

17. A deodorizing device, said deodorizing device comprising:
a cylindrical body having a first end and a second end;
a mouthpiece, said mouthpiece is removably attached to said first end of said cylindrical body;
a first cartridge having a first end and a second end, said first cartridge being housed in said cylindrical body;
a second cartridge, said second cartridge having a first end and a second end, said second cartridge being housed in said cylindrical body and removably coupled to said first cartridge;
a first filter, said first filter being housed in said cylindrical body and being removably attached to said first end of said first cartridge;
a second filter, said second filter being housed in said cylindrical body and being removably attached to said second end of said first cartridge;
a plurality of seals said plurality of seals being housed in said cylindrical body;
a screen, said screen being removably coupled to said second end of said cylindrical body;
wherein said deodorizing device is configured to deodorize an odor;
wherein said first cartridge holds charcoal;
wherein said second cartridge holds an extract;
wherein said extract is an organic fruit extract;
wherein said charcoal is activated charcoal;
wherein said activated charcoal is in powder-form;
wherein said second cartridge is removably coupled to said first cartridge by a first threading means;
wherein said first and said second cartridges are replaceable;
wherein said first and said second filters are made of foam;
wherein said mouthpiece is removably attached to said first end of said cylindrical body by a second threading means;
wherein said plurality of seals includes a first seal and a second seal;
wherein said first seal is removably attached to said first end of said first cartridge;
wherein said second seal is removably attached to said screen;
wherein said plurality of seals are made of rubber;
wherein said plurality of seals are O-ring seals; and
wherein said odor includes medical marijuana.
